(19) United States
(12) Patent Application Publication
(10) Pub. No.: US 2011/0155144 A1
(43) Pub. Date: Jun. 30, 2011

(54) ADJUSTABLE MANDIBULAR PROTRUSION SPLINT FOR TREATING SNORING AND OBSTRUCTIVE SLEEP APNEA

(76) Inventor: Winfried Tousssaint, Bensheim (DE)

(21) Appl. No.: 13/060,304

(22) PCT Filed: Aug. 21, 2009
(86) PCT No.: PCT/DE09/01179

§ 371 Date: Feb. 23, 2011

(30) Foreign Application Priority Data
Sep. 6, 2008 (DE) 20 2008 011 841.7

(51) Int. Cl. A61F 5/56; B23P 17/00

(52) U.S. Cl. 128/848; 29/527.1

(57) ABSTRACT

A two-part mandibular protrusion splint (1) for preventing at least one of snoring and obstructive sleep apnea. The mandibular protrusion splint (1) comprises both a lower part (2) and an upper part (3) and each part includes a shaping and flexurally rigid tray (2a, 3a) which is open, during use, towards the mandibula and the maxilla. Each tray comprises a thermoplastic filler material (4, 5) which can be shaped in a manner of a dental brace to fit the human maxilla and mandibula. Both the trays comprise one or more fixing knobs (5a, 5b, 6a, 6b, 7a, 7b, 8a, 8b), on the respective outer sides thereof, for fastening to a protrusion holder (9). The protrusion holder (9) is rotatably fastened to a fixing knob (5a, 5b, 6a, 6b, 7a, 7b, 8a, 8b), on each of the mandibular tray and the maxillar tray, for bring the mandibula into an anterior position.

### PRIORITY APPLICATION

The complete disclosure of the underlying utility model application DE 20 2008 011 of 06.09.2008, which priority is claimed herein is hereby incorporated by reference.

### BACKGROUND OF THE INVENTION

1. Technical Field

The invention relates to an adjustable two-part mandibular protrusion splint for the treatment of snoring and/or obstructive sleep apnea.

2. Prior Art

Snoring may be a symptom of obstructive sleep apnoea syndrome, characterized by repeated and frequent cessation of breathing at night, which may be associated with severe health complications, such as, for example, high blood pressure, cardiovascular diseases, strokes, inter alia. U.S. Pat. No. 5,462,066 and European patent application EP 1 203 570 have disclosed such brace-like bite splints for preventing snoring, which serve to move the mandibula slightly forwards, since in this position of the mandibula the respiratory tracts are opened wider, enabling the patient to breathe more freely without snoring.

The known bite splints in form of a one-piece, brace-like mouthpiece consist of thermoplastic materials having two bite channels, which become shapeable upon warming. The patient puts the warmed, yet unfitted mouthpiece in the mouth and then presses the mandibular and maxillar teeth into the corresponding lower and upper bite channels in the shapeable plastic and fits them to the bite channels by biting on the bite plates. The plastic cools in the process and regains its solid elasticity, after which the mouthpiece has been fitted to the patient. During the fitting operation, it must be ensured that the mandibula is moved forwards somewhat in order to establish a permanent protrusion. The known bite splints have the disadvantage that a protrusion, once established, can only be adapted with difficulty to changing needs of the patient, and consequently the effect achieved initially deteriorates with time.

Furthermore, U.S. Pat. No. 5,868,138 proposes a dental appliance for treating snoring and obstructive sleep apnoea which has a maxillar part, a mandibular part and a connecting means, where the connecting means retains the lower part in an adjustable manner in an anterior, protruded position relative to the upper part.

The German utility model DE 29 506 512 proposes an orally bearable anti-snoring device comprising a maxillar and a mandibular splint, wherein both the splints are provided with a flexible, in the longitudinal direction inextensible tension, which brings the mandible to an anterior position upon lowering the same.

For this purpose, maxillar and mandibular splints have to be manufactured individually in the dental laboratory after an impression has been taken.

The present invention was based on the object of providing snorers and sleep apnoea sufferers with a mandibular protrusion splint with comparable efficacy which has compared to this a long service life and is easy to handle. Furthermore, the mandibular protrusion splint of the invention should be easily adaptable by the patient himself, which requires a significant simplification in handling to the requirements. In addition, the advancement of the lower jaw by the mandibular protrusion splint according to this invention should be adaptable to the requirements of the patient.

This problem has been solved by providing a two-piece mandibular protrusion splint comprising a lower (2) and an upper part (3), which include shaping and flexurally rigid trays (2a, 3a) which are open towards the mandibula and the maxilla, respectively, during use, each comprising a thermoplastic filler material (4, 5) which can be shaped in each case in the manner of a dental brace to fit the human maxilla and mandibula, wherein the two trays comprise one or more fixing knobs on the respective outer sides thereof for fastening a protrusion holder, which is rotatably fastened to a fixing knob each on the mandibular tray and the maxillar tray and brings the mandibula into an anterior position. With the aid of several protrusion holders of different lengths the mandibular advancement can easily be customized to the requirements of each user.

### BRIEF SUMMARY OF THE INVENTION

The invention thus relates to a general-purpose two-part mandibular protrusion splint (1) for the prevention of snoring and/or obstructive sleep apnea, comprising a lower (2) and an upper part (3), which include shaping and flexurally rigid trays (2a, 3a) which are open towards the mandibula and the maxilla, respectively, during use, each comprising a thermoplastic filler material (4, 5) which can be shaped in each case in the manner of a dental brace to fit the human maxilla and mandibula, wherein the two trays comprise one or more fixing knobs on the respective outer sides thereof for fastening a protrusion holder, which is rotatably fastened to a fixing knob each on the mandibular tray and the maxillar tray and brings the mandibula into an anterior position.

The mandibular protrusion splint according to the invention avoids the time-consuming and costly individual manufacturing in the dental laboratory with prior dental impression to be taken by a dentist or orthodontist. Moreover, this universal splint can easily be adjusted by any doctor—not only by dentists or even by the patient himself, resulting in a significant simplification in the handling requires.

Furthermore, the invention relates to a process for producing a novel mandibular protrusion splint comprising the steps of

(a) forming of the shaping and rigid trays (2a, 3a) each having preformed fixing knobs on the outer sides by an injection molding process,
(b) filling the trays with a thermoplastic filler material (4, 5);
(c) forming of the protrusion holders;
(d) connecting the protrusion holders with the preformed fixing knobs. Furthermore, the invention relates to the use of the two-piece mandibular protrusion splint (1) for producing a device for preventing snoring and/or obstructive sleep apnea.

Another object of the invention is a ready-to-use set for preparing a novel mandibular protrusion splint to prevent snoring and/or (obstructive) sleep apnea consisting of

(A) a lower (2) and an upper part (3), which include shaping and flexurally rigid trays (2a, 3a) which are open towards the mandibula and the maxilla, respectively, during use, each comprising a thermoplastic filler material (4, 5) which can be shaped in each case in the manner of a dental brace to fit the human maxilla and mandibula, wherein the two trays comprise one or more fixing knobs on the respective outer sides thereof for fastening a protrusion holder, which upon lowering the mandible brings it in dependence of the length of the protrusion holders in an anterior or posterior position;
(B) two sets of two or more protrusion holders (10) of different length.

The inventive universal mandibular protrusion splint has the advantage that it is very firmly attached or adhered on both jaws like a dental splint, since a very deep and uniform impression of all the teeth is achieved without any problems due to the material properties of the thermoplastic filler of the splint. The tightness is favored by the fixed frame of the thermoplastic by the solid outer walls of a rigid material consisting of, for example, polycarbonate. Furthermore, the mandibular protrusion splint has the advantage that it can be adapted easily without special equipment by any doctor or even by the patient himself within few minutes. For this reason, one universal standard splint suffices to fit for almost all jaw forms. Furthermore, an individual adjustment of the mandibular advancement is achievable in the most advantageous way by the use of protrusion holders of different lengths. Due to the special construction of the splint one achieves that the same is very delicate and the distance between the upper and lower front teeth with less than 3 mm is very small upon adjustment, which has a very positive effect on comfort and acceptance and that the splint is suitable for different sizes of jaw formations at the same time.

### BRIEF DESCRIPTION OF THE DRAWINGS

FIG. 1 shows a perspective view at an angle from the top and anterior of an embodiment of the universal protrusion splint according to the invention (1);

FIG. 2 shows a schematic depiction of an unfilled tray for the upper jaw (2a);

FIG. 3 shows a schematic depiction of a filled tray for the upper jaw (2a);

FIG. 4 shows a top view of the upper part from above;

FIG. 5 shows a schematic side view of the upper part;

FIG. 6 shows a schematic view of a protrusion holder;

FIG. 6a shows a section along the line drawn in FIG. 6 from “A” to “A”.

FIG. 6b shows a section along the line drawn in FIG. 6 from “B” to “B”.

FIG. 7 shows a schematic view of a set of 8 protrusion holders of different lengths, which are mounted in a trapezoid-shaped frame.

FIG. 8 shows a perspective view at an angle from the top and anterior an alternative embodiment of the universal protrusion splint (1);

FIG. 9 shows a top view of the upper part from the top of the alternative embodiment of the inventive universal protrusion splint (1),

FIG. 10 shows a schematic view of a set of 6 protrusion holders of different lengths, which are mounted in a trapezoid-shaped frame for the alternative embodiment of the universal protrusion splint according to the invention.

### DETAILED DESCRIPTION OF THE INVENTION

The protrusion splint according to the invention can be manufactured from known materials in an universally mass production fasion by injection molding.

The term “protrusion splint” or “mandibular protrusion splint” as it is used hereinbefore and hereinbelow refers to a dental device that allows to move the lower jaw slightly forward in relation to the upper jaw into an advanced position and thereby to increase the cross-section of the upper respiratory tract. This leads to a reduction of snoring and of breathing interruptions due to an obstructive sleep apnea.

The term “thermoplastic filler material” as it is used hereinbefore and hereinbelow refers to a material that can be plastically deformed in the heat, preferably below of 70° C., preferably between 40 and 65° C. and closely adapts to a specified form and upon cooling maintains the said form. Suitable materials are amongst others for example polymers and copolymers or mixtures thereof from the group of polyethylenes (PE), polyvinyl acetates (PVA), acrylates and methacrylates, preferably copolymers of PE and PVA, such as those which are available under the trademark Elvax®® by the company DuPont. Preferred are such PE-PVA copolymers, wherein the PVA content is from 20 to 40, in particular, from 25 to 35%.

The term “shaping and rigid tray” as it is used above and below refers to a “U- or horseshoe-shaped” mold, which forms at its ends an open or closed tray or tub. As a rule, this mold consists of a material which is stable and inert under physiological conditions such as thermosetting plastics for example polytetrafluoroethylene (PTFE, Teflon®®) or polycarbonates, or of elastomers, such as polyacrylates.

The term “fixing knob” as it is used hereinbefore and hereinbelow refers to a rod-shaped elevation with a terminal thickening, each of which is attached to the side of the shell for the lower or upper jaw, respectively, and to which the protrusion holder can be attached reversibly. As a rule, the fixing knob is designed in a way that the terminal thickening snaps into a recess of the protrusion holder and fastens it rotatably around the longitudinal axis of the fixing knob.

The term “protrusion holder” as it is used hereinbefore and hereinbelow refers to a connecting element between the trays of the upper and the lower jaw, which enables that the lower jaw is brought into an anterior position when it is applied. It exhibits terminal recesses, in each of which a fixing knob can engage. The geometry of the protrusion holder is not critical per se; it should be long enough to bring the lower jaw in an anterior position. It should be stable towards stretching or compression. It must be relatively thin to ensure a good wearing comfort.

The above and below used indications regarding the geometry or spatial arrangement of the protrusion splint or parts of it are based on the arrangement of the protrusion splint in its reasonably intended use, i.e. after the introduction of the same into the upper and lower teeth of the affected person: “in the longitudinal direction” means towards the mouth opening (“anterior”) or throat (“posterior”), “forward” (anterior) means in the direction of the mouth, “centered” means in the area of the incisors, “back” means in the region of the posterior molars; “lateral” means the region between the premolars and the posterior molars.

Advantageous embodiments of the invention are such mandibular protrusion splints according to the invention, wherein

(a) the fixing knobs exhibit a diameter from 2.0 to 5.0 mm, preferably from 3.0 to 4.5 mm, in particular about 4 mm;
(b) the distance between the inside of the bottom of the maxillary tray (11) and the inside of the bottom of the mandibular tray (12), in the assembled state is less than 8 mm, preferably 1 to 5 mm, in particular about 2 mm;
(c) the distance of the teeth in the introduced splint in the anterior region is about 2 to 4 mm. Particularly preferred is a distance of the teeth in the anterior region of about 3 mm with the introduced splint. As a rule, this leads usually to a easier and more complete closure of the mouth even with small sized jaws without muscle tension. This results in a greater comfort and hence a better acceptance (compliance).
(d) the filling material (4, 5) consists of one or more copolymers of polyethylene and polyvinyl acetate;
(e) the shaping and rigid tray (2a, 3a) is made of polycarbonate;
(f) the protrusion holder is made of an elastic and tear-resistant material, in particular of polyoxymethylene (POM, e.g. Ultraform/BASF);

Preferably, the protrusion holders, whose lengths is shorter than the distance between two fixing knobs, are each connected to the front fixing knob of the upper part and the rear fixing knob of the lower part, wherein the jaw is brought by tension into an anterior position, preferably wherein the rigid trays (2a, 3a) each exhibit at its two outer sides, two fixing knobs in a distance of 20.0 to 30.0 mm, preferably of 22.0 to 28.0 mm, in particular of about 26.7 mm, and/or the protrusion holders exhibit a total length from 15.0 to 35.0 mm, preferably from 17.0 to 31.0 mm.

In an alternative embodiment, the protrusion holder, whose length is longer than the distance between two fixing knobs, are each connected to the rear fixing knob of the upper part and the front fixing knob of the lower part, whereby the lower jaw is moved by pressure into an anterior position, preferably with the rigid trays (2a, 3a) each exhibit at its two outer sides, two fixing knobs in a distance of 10.0 to 20.0 mm, preferably of 12.5 to 18.0 mm, in particular of about 15.6 mm and/or the protrusion holders the exhibit a total length from 15.0 to 30.0 mm, preferably from 17.0 to 27.0 mm.

The manufacture of the universal mandibular protrusion splint per se is simple:

(a) one forms the shaping and rigid trays (2a, 3a) each having on the outside preformed fixing knobs by an injection molding process,
(b) one fills the trays with a thermoplastic filler material (4, 5), also by injection molding for mass production or in special cases, also manufactured manually made by hand, and
(c) one forms the necessary protrusion holders each in pairs, and
(d) one connects the protrusion holders with the preformed fixing knobs. The used polymer or copolymer of the thermoplastic filling material can also in special cases be, apart from the usual mass-produced by injection molding, after heating in granular form in a water bath manually transformed into a homogeneous plastic mass and then be pressed into the empty unfilled trays. If one then heats the trays with their filling in hot, preferably boiling water for about 2 to 4 minutes and upon short cooling, distributes the plastic material evenly into the trays and then hardens it, one can produce a fully functional mandibular protrusion splint according to the invention. The polymeric mass adheres absolutely firm on the contact surface of polycarbonate. In the event that a doctor or a patient fails the adaptation at the first attempt due to biting at the wrong place, the splint can be restored into the original state very easily in this way.

In the event that the adhesion to the teeth and jaws decrease after a long wearing period of many months, one can thus also easily restore the original splint filling completely and then repeat the adjustment. The mandibular protrusion splint according to the invention can therefore be used optionally upon interim repairs for a very long period of time.

Examples of the invention are shown in the drawings and are described in more detail as follows.

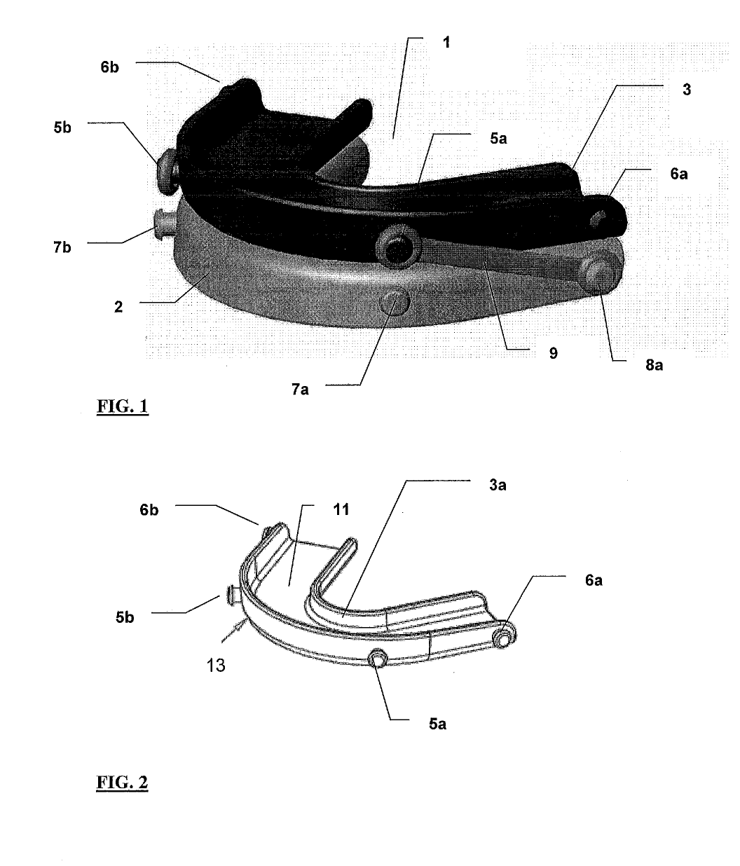

FIG. 1 shows a perspective view at an angle from the top and anterior of an embodiment of the universal protrusion splint according to the invention (1), which is formed from a lower part (2) and an upper part (3), each consisting of a tray (2a, 3a) and a filling material (4), which is not shown. Furthermore, both the lower part (2) and the upper part (3) of the protrusion splint (1) each have two fixing knobs (5a, 6a, 7a, 8a, 5a, 6b, 7b, 8b) at their two side areas, wherein the two front fixing knobs of the upper part (5a, 5b) each have a protrusion holder (9), which are connected to the two rear fixing knobs (8a, 8b) of the lower part.

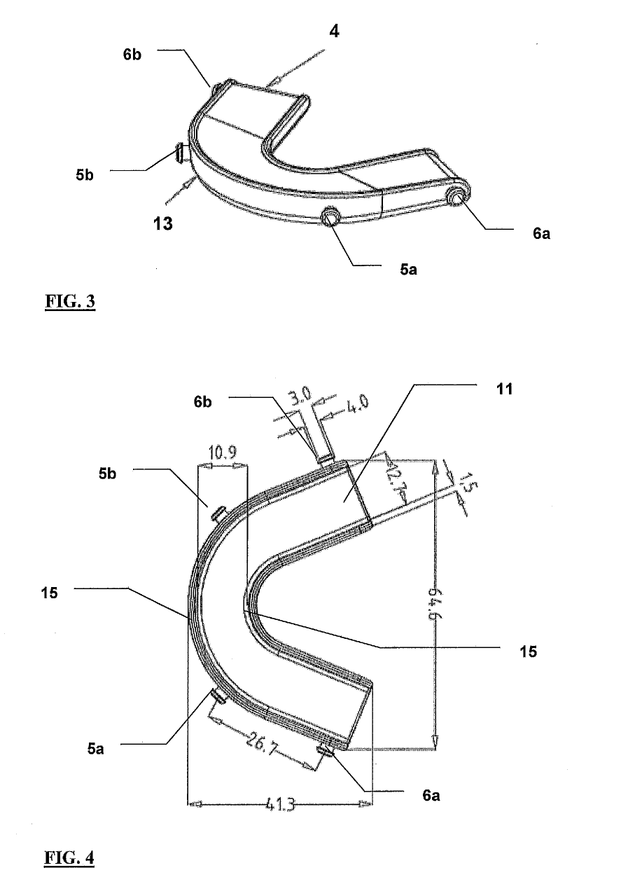

FIG. 2 shows a schematic representation of an unfilled maxillary tray (3 a), with the four fixing knobs (5a, 6a, 5b, 6b). As can be seen from FIG. 3, the thermoplastic material (4) surmounts in each case the height of the flexurally rigid trays, preferably by 0.5 to 3 mm, in particular by 1 to 1.5 mm. This results in an increased adhesion of the teeth, and in a protection of the gums against injury from the edges of the flexurally rigid trays.

FIG. 4 shows a top view of the upper part (3) from above, in which the dimensions of the different areas of the upper shell are indicated. For instance, the distance between the two outer walls (15) of the tray at the posterior side is 12.7 mm and 10.9 mm in the anterior region. The wall thickness of the outer wall (15) is about 1.5 mm. The distance between the foremost position of the tray and the very back position is approximately 41.3 mm and the distance between both the posterior ends of the outer shell is about 64.6 mm. The distance between the front fixing knob (5a, 5b) and the respective posterior fixing knob (6a, 6b) is about 26.7 mm.

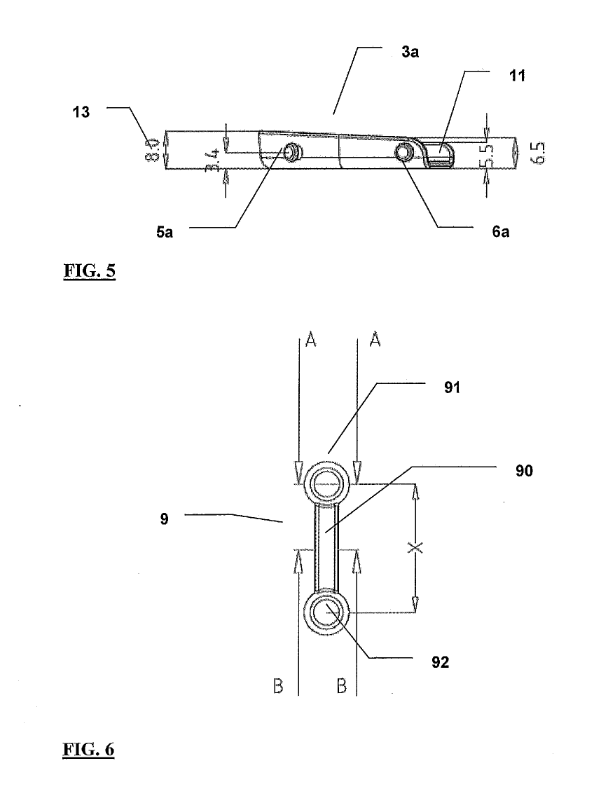

FIG. 5 shows a schematic side view of the upper part (3a), wherein the height of the outside wall (15) is indicated. This is about 8 mm in the anterior region, and about 5.5 to 6.5 mm in the posterior region.

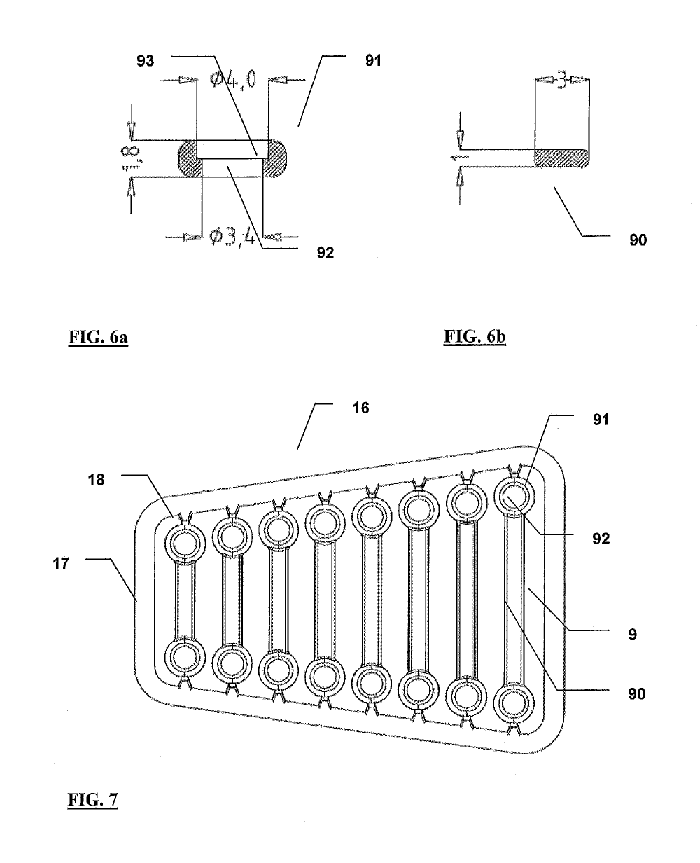

FIG. 6 shows a schematic view of a protrusion holder (9) with both the terminal fastening elements (91) and their ring-shaped recesses (92).

FIG. 6a shows a sectional view along the line “A” to “A” drawn in FIG. 6 through a fastening element (91) and indicates the respective dimensions of the same. One clearly recognizes the gradation (93) of the annular recess (92) that enables a better latching of the fastening element (91) into one of the fixing knobs. The fastening element is about 1.8 mm thick and the annular recess exhibits an inner diameter of about 3.4 mm at the respective side towards the tray and an inner diameter of about 4.0 mm at the side facing away therefrom.

FIG. 6b shows a sectional view along the line drawn from “B” to “B” in FIG. 6 through the fastening element (91) and indicates the respective dimensions of the latter, with the fastening element (91) being about 1 mm thick and about 3 mm wide.

FIG. 7 shows a schematic view of a set (16) of 8 protrusion holders (9) of different lengths, which are mounted in a trapezoid-shaped frame (17) by fasteners (18). The particular required protrussion holder can easily be removed from the holder by turning or breaking.

FIG. 8 shows a perspective view at an angle from the top and anterior of an alternative embodiment of the universal protrusion splint (1) which is formed from a lower part (2) and an upper part (3), each consisting of a tray (2a, 3a) and non-illustrated filler (4). Furthermore, both the lower part (2) and the upper part (3) of the protrusion splint (1) each have at both the side areas, two fixing knobs (5a, 6a, 7a, 8a, 5a, 6b, 7b, 8b) wherein the two rear fixing knobs of the upper part (6a, 6b) each are connected by a protrusion holder (9) with the two front fixing knobs (7a, 7b) of the lower part.

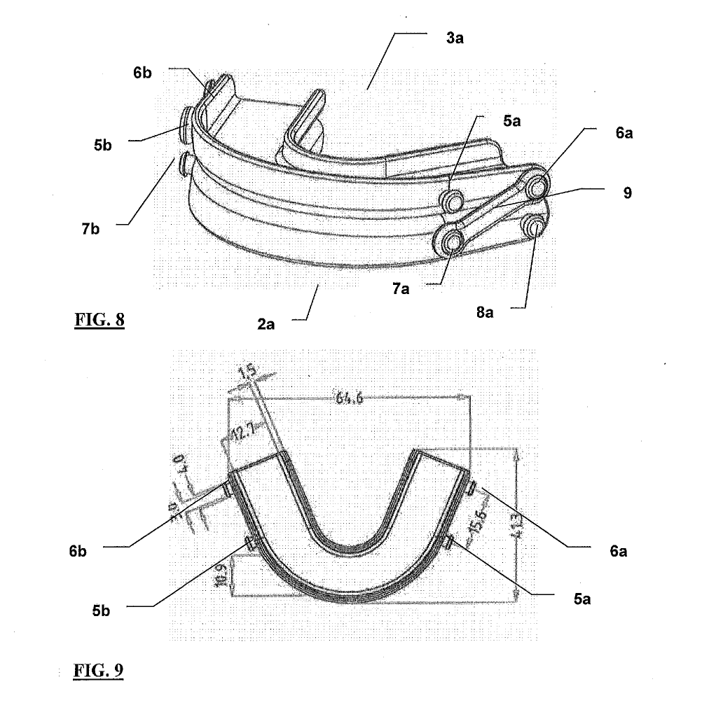

FIG. 9 shows a top view of the upper part of the alternative embodiment of the universal protrusion splint (1) of FIG. 8, wherein the dimensions of the different areas of the upper tray are indicated. It is evident that the distances between the front fixing knobs (5a, 5b) and the rear fixing knobs (6a, 6b) of this embodiment are shorter, namely only about 15.6 mm, than in the embodiment of FIG. 3.

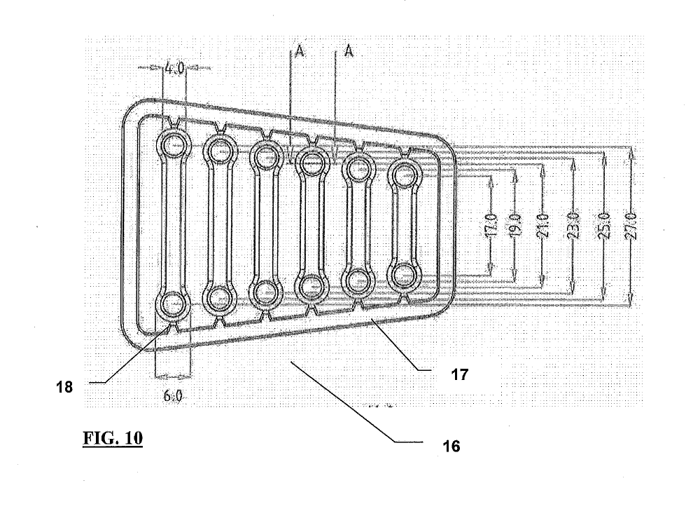

FIG. 10 shows a schematic top view of a set of 6 protrusion holders of different lengths, which are mounted in a trapezoid-shaped frame for the alternative embodiment of the universal protrusion splint according to the invention.

The distance between the inner bottom of the two trays in front teeth area (“tooth gap”) is less than 8 mm, preferably 1 to 5 mm, in particular about 2 mm. The protrusion splint exhibits before adjustment to the teeth a total height of less than 20 mm, preferably 10 to 20 mm, particularly about 16 mm in the area of the incisors.

The detailed views of the upper part (3) shown in the FIG. 2 to 5 and 9 illustrate the structure and function of this part of the protrusion splint according to the invention.

The distance between both the outer walls at the rear end of the upper part (maximum width of the mandibular splint) is 60 to 70 mm, preferably 62 to 68 mm, particularly about 65 mm. The distance between the outer wall (33) in the area of the incisors and an fictive line between the rear ends of the inner wall (43) (maximum length of the mandibular splint) is 38 to 45 mm, preferably about 41.3 mm (cp. FIG. 9). 3.0 to 4.0 cm³ of polycarbonate are used, in order to prepare the flexurally rigid upper tray (3).

5.0 to 12.0 cm³ of a copolymer of PE and PVA are used, in order to prepare the filling of the upper shell.

The adjustment of the universal protrusion splint according to the invention to the jaw of the person in need thereof is simple. In general the adjustment may be carried out by any doctor of any specialty, or his instructed personnel or in most cases by the person in need thereof alone with the aid of a mirror.

In the case of malposition of the teeth, anamnestic jaw joint disorders or dental diseases, such as periodontal disease, it is advisable to consult a dentist before the adjustment.

In adapting the splint according to the invention the upper part (3) is heated first, to a temperature above the softening temperature of the filler (4), preferably in a water bath at a water temperature above 50° C. Thereafter one fits the upper part to the teeth of the upper jaw by pressing the teeth evenly deep into the soft, plastic and still warm filling material. Subsequently the filling material is allowed to cool down for just about 30-60 seconds in the mouth and then finally cured in a cold water bath.

Then one heats to the lower part (2) accordingly, and connects it with the already adapted upper part with aid of the two protrussion holder of the same length, one puts the adapted upper part into the upper jaw and presses the teeth of the lower jaw gently into the soft filling material (4) of the lower part. After curing, the pre-mandibular is operational.

The mandibular protrusion splint according to the invention eliminates or reduces snoring and cessation of breathing due to obstructive sleep apnea. Due to its delicate size, it exhibits a comfortable fit. It provides a solid, secure hold for the embraced teeth, can be adjusted by replacing the protrusion holder in small stages (incremental) in order to adapt the mandibular advancement to the needs of the patient, it protects the teeth against nocturnal bruxism and the gum against injuries.

1-21. (canceled)

22. A two-part mandibular protrusion splint (1) for preventing at least one of snoring and obstructive sleep apnea, the mandibular protrusion splint (1) comprising:
a lower part (2) and an upper part (3), the lower part (2) including a shaping and flexurally rigid mandibular tray (2a) and the upper part (3) including a shaping and flexurally rigid maxillar tray (3a) which both respectively open toward a mandibula and a maxilla, respectively, during use; and
each of the rigid mandibular and maxillar trays (2a, 3a) respectively comprising a thermoplastic filler material (4, 5) which can be shaped in a manner of a dental brace for respectively fitting to the maxilla and the mandibula;
wherein each of the rigid mandibular and maxillar trays (2a, 3a) comprise at least one fixing knob, on an outer surface thereof, for fastening a protrusion holder (9), whereby the protrusion holder (9) is rotatably fastened to a fixing knob of each of the rigid mandibular tray (2a) and the rigid maxillar tray (3a) for bring the mandibula into an anterior position.

23. The mandibular protrusion splint according to claim 22, wherein each of the at least one fixing knobs has a diameter of between 2.0 and 5.0 mm.

24. The mandibular protrusion splint according to claim 23, wherein the diameter of each of the at least one fixing knob is between 3.0 and 4.5 mm.

25. The mandibular protrusion splint according to claim 22, wherein a distance between an inside of a bottom of the rigid maxillary tray (23) and an inside of a bottom of the rigid mandibular tray (22), in an assembled state, is less than 8 mm.

26. The mandibular protrusion splint according to claim 25, wherein the distance in the assembled state is between 1 and 5 mm.

27. The mandibular protrusion splint according to claim 25, wherein, with the mandibular protrusion splint in place, the distance of the teeth in an anterior tooth region is between 2 and 4 mm.

28. The mandibular protrusion splint according to claim 22, wherein the filling material (4, 5) is one of a biocompatible toxicologically acceptable polymer and a biocompatible toxicologically acceptable copolymer which is plastically deformable, when heated, so that the one of the biocompatible toxicologically acceptable polymer and the biocompatible toxicologically acceptable copolymer can closely adapt to a specified form and then retain the specified form upon cooling.

29. The mandibular protrusion splint according to claim 28, wherein the filling material (4, 5) is plastically deformable below 70° C.

30. The mandibular protrusion splint according to claim 22, wherein the rigid mandibular and maxillar trays (2a, 3a) each comprise a polycarbonate.

31. The mandibular protrusion splint according to claim 22, wherein the protrusion holders (9) each comprise an elastic and tear-resistant plastic material.

32. The mandibular protrusion splint according to claim 22, wherein the protrusion holders (9) each comprise polyoxymethylene.

33. The mandibular protrusion splint according to claim 22, wherein the protrusion holders, whose lengths are shorter than a distance between two fixed knobs, are each connected with a front fixing knob of the rigid maxillar tray (3a) and a rear fixing knob of the rigid mandibular tray (2a) so that the lower jaw is placed into the anterior position by tension.

34. The mandibular protrusion splint according to claim 33, wherein each of the rigid mandibular and maxillar trays (2a, 3a) have two fixing knobs, on an outer surface thereof, which are space apart by a distance between 20.0 and 30.0 mm.

35. The mandibular protrusion splint according to claim 34, wherein the distance is between 22.0 and 28.0 mm.

36. The mandibular protrusion splint according to claim 34, wherein each protrusion holder has a total length of between 15.0 and 35.0 mm.

37. The mandibular protrusion splint according to claim 22, wherein the protrusion holders, whose lengths are longer than the distance between two fixing knobs, are each connected with the rear fixing knob of the rigid maxillar tray (3a) and the front fixing knob of the rigid mandibular tray (2a), whereby the lower jaw is placed by pressure into an anterior position.

38. The mandibular protrusion splint according to claim 37, wherein the rigid mandibular and maxillar trays (2a, 3a) each have two fixing knobs on an outer surface thereof spaced apart by a distance of between 10.0 and 20.0 mm.

39. The mandibular protrusion splint according to claim 38, wherein the distance is between 12.5 and 18.0 mm.

40. The mandibular protrusion splint according to claim 38, wherein each protrusion holder has a total length between 15.0 and 30.0 mm.

41. The mandibular protrusion splint according to claim 22, wherein each protrusion holder has, at both ends thereof, an annular recess for receiving a respective fixing knob on the rigid mandibular and maxillar trays (2a, 3a).

42. The mandibular protrusion splint according to claim 41, wherein the annular recess, for receiving a respective fixing knob, has an inner diameter of between 3.0 and 4.5 mm.

43. The mandibular protrusion splint according to claim 41, wherein the annular recess is step like and has, on one side, an inner diameter of between 3.8 and 4.2 mm and, on the other side, an inner diameter of between 3.1 mm and 3.6 mm.

44. A method for producing a mandibular protrusion splint for preventing at least one of snoring and obstructive sleep apnea, the mandibular protrusion splint (1) comprising a lower part (2) and an upper part (3), the lower part (2) including a shaping and flexurally rigid mandibular tray (2a) and the upper part (3) including a shaping and flexurally rigid maxillar tray (3a) which both respectively open toward a mandibula and a maxilla, respectively, during use; and each of the rigid mandibular and maxillar trays (2a, 3a) respectively comprising a thermoplastic filler material (4, 5) which can be shaped in a manner of a dental brace for respectively fitting to the maxilla and the mandibula; wherein each of the rigid mandibular and maxillar trays (2a, 3a) comprise at least one fixing knob, on an outer surface thereof, for fastening a protrusion holder (9), whereby the protrusion holder (9) is rotatably fastened to a fixing knob of each of the rigid mandibular tray (2a) and the rigid maxillar tray (3a) for bring the mandibula into an anterior position, the method comprising the steps of:
(A) forming the rigid mandibular and maxillar trays (2a, 3a) by an injection molding process with each of the rigid mandibular and maxillar trays (2a, 3a) having, on the outside thereof, preformed fixing knobs,
(B) filling the trays with the thermoplastic filler material (4, 5);
(C) forming the protrusion holders; and
(D) connecting the protrusion holders with the preformed fixing knobs.

45. A ready-to-use set for producing of a mandibular protrusion splint and preventing at least one of snoring and (obstructive) sleep apnea, the ready-to-use set comprising:
(A) a lower part (2) and an upper part (3), the lower part (2) including a shaping and flexurally rigid mandibular tray (2a) and the upper part (3) including a shaping and flexurally rigid maxillar tray (3a) which both respectively open toward a mandibula and a maxilla, respectively, during use; and each of the rigid mandibular and maxillar trays (2a, 3a) respectively comprising a thermoplastic filler material (4, 5) which can be shaped in a manner of a dental brace for respectively fitting to the maxilla and the mandibula; wherein each of the rigid mandibular and maxillar trays (2a, 3a) comprise at least one fixing knob, on an outer surface thereof, for fastening a protrusion holder (9), whereby the protrusion holder (9) is rotatably fastened to a fixing knob of each of the rigid mandibular tray (2a) and the rigid maxillar tray (3a) for bring the mandibula into an anterior position; and
(B) at least a first set of at least two protrusion holders (10) and a second set of at least two protrusion holders (10), and the first set of at least two protrusion holders (10) has a different length than the second set of the at least two protrusion holders (10).

46. The ready-to-use set according to claim 45, comprising between 3 and 10 sets of at least two protrusion holders (10) which each have different lengths, and a set of a shortest protrusion holder has a length of 17.0 mm while a set of a longest protrusion holder has a length of 31.0 mm.

47. The ready-to-use set according to claim 46, wherein the protrusion holders of different lengths are assembled together within a frame.

48. The mandibular protrusion splint according to claim 22, wherein the protrusion holders (9) each are stable with respect to one of stretching and compression.
